(19) United States
(12) Patent Application Publication
(10) Pub. No.: US 2021/0386940 A1
(43) Pub. Date: Dec. 16, 2021

(54) Body Limb Support System

(71) Applicant: David Anthony Nicholson, Rome, GA (US)

(72) Inventor: David Anthony Nicholson, Rome, GA (US)

(21) Appl. No.: 17/344,865

(22) Filed: Jun. 10, 2021

Related U.S. Application Data
(60) Provisional application No. 63/037,368, filed on Jun. 10, 2020.

(51) Int. Cl. A61M 5/52; F16M 11/28; F16M 11/24; A61M 37/00

(52) U.S. Cl. CPC A61M 5/52; A61M 37/0076; F16M 11/242; F16M 11/28

(57) ABSTRACT

A body limb support system is a system that can be used to prop up different body parts during a tattoo session for greater comfort to the user and the tattoo artist and to facilitate the procedure. The system may include at least one limb pad. The at least one limb pad is a versatile support device that can be arranged in multiple ways to support different body areas. The at least one limb pad may include a pad body and a plurality of pad fasteners. The pad body is preferably a soft structure that supports the target body area without causing damage to the body limb or the surface onto which the pad body is placed. The plurality of pad fasteners enables multiple units of the at least one limb pad to be attached to each other in different configurations to support the target body area during the procedure.

The current application claims a priority to the U.S. provisional patent application Ser. No. 63/037,368 filed on Jun. 10, 2020.

### FIELD OF THE INVENTION

The present invention relates generally to body support equipment. More specifically, the present invention is a support system that can be used to support portions of a client's body during a tattoo session.

### BACKGROUND OF THE INVENTION

Tattoos have been part of human culture for thousands of years. Nowadays, tattoos are largely considered body art and are widely popular, with greater interest found in younger people and slightly more in women. The increasing popularity of tattoos has inspired refinement in tattoo equipment used to facilitate the tattooing process, especially equipment that provides comfort during the tattoo session. For example, a typical tattoo studio may have a variety of tools including, but not limited to, a tattoo chair, table, armrest, footrest, hip rest, etc. However, several drawbacks are associated with the existing equipment currently used in the tattoo industry to support body parts during the tattoo session. The tattoo studio is required to keep different tools to accommodate different body parts, which take up space. Moreover, some tools are not specifically designed to be used to apply tattoos, but rather, are used for other applications, such as in medical or beauty salon applications. Tattoo artists may sometimes find it difficult to position a client's body parts, such as arms, legs, or foot using these devices. In addition, the use of such tools in tattooing may be uncomfortable for both the tattoo artist and the client. The present invention aims to solve some of these problems by disclosing a new and improved body limb support system.

### SUMMARY OF THE INVENTION

The present invention is a body limb support system designed to help users prop up different body parts during a tattoo session. The present invention provides multiple limb pads that can be used as a footrest, a leg rest, an armrest, a hip rest, etc. The present invention further provides a support stand that can be used to elevate the limb pads to support the desired body part. The support stand, preferably referred to as the “Last Stand,” is a durable accessory that can be provided along with the limb pads. Other additional accessories, such as a support strap, are provided that can be used with the limb pads. The multiple limb pads, the support stand, and that support strap can be provided together as a customizable kit. Alternatively, each component can be provided separately or as a combination of the three. While the present invention is preferably used during tattoo sessions, the present invention is a versatile support system that provides a support surface onto which a body part or an object can be placed for different applications. Additional benefits and features of the present invention are further discussed in the sections below.

### BRIEF DESCRIPTION OF THE DRAWINGS

FIG. 1 is a top perspective view showing the at least one limb pad of the present invention.

FIG. 2 is a bottom perspective view showing the at least one limb pad of the present invention.

FIG. 3 is a front view showing the at least one limb pad of the present invention.

FIG. 4 is a cross-sectional view of the pad body taken along line 4-4 in FIG. 3.

FIG. 5 is a top perspective view showing the at least one limb pad, wherein the at least one limb pad is shown as a first limb pad and a second limb pad.

FIG. 6 is a top view showing the first limb pad and the second limb pad arranged in a first configuration.

FIG. 7 is a side view showing the first limb pad and the second limb pad arranged in a second configuration.

FIG. 8 is a side view showing the first limb pad and the second limb pad arranged in a third configuration.

FIG. 9 is a top perspective view showing the first limb pad and the second limb pad arranged in a fourth configuration.

FIG. 10 is a top view showing the at least one support strap fastened to the at least one limb pad.

FIG. 11 is a top perspective view showing the support stand of the present invention.

FIG. 12 is a bottom perspective view showing the support stand of the present invention.

FIG. 13 is a top perspective view showing the at least one limb pad being mounted onto the foldable platform of the support stand.

FIG. 14 is a top perspective view showing the at least one limb pad mounted onto the foldable platform of the support stand.

FIG. 15 is a side view showing the support stand of the present invention in a collapsed configuration.

FIG. 16 is a top perspective view showing the plurality of pad fasteners of the present invention.

### DETAIL DESCRIPTIONS OF THE INVENTION

The foregoing and other objects and advantages will appear from the description to follow. It is to be understood, however, that the present invention may be embodied in various forms. These embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense.

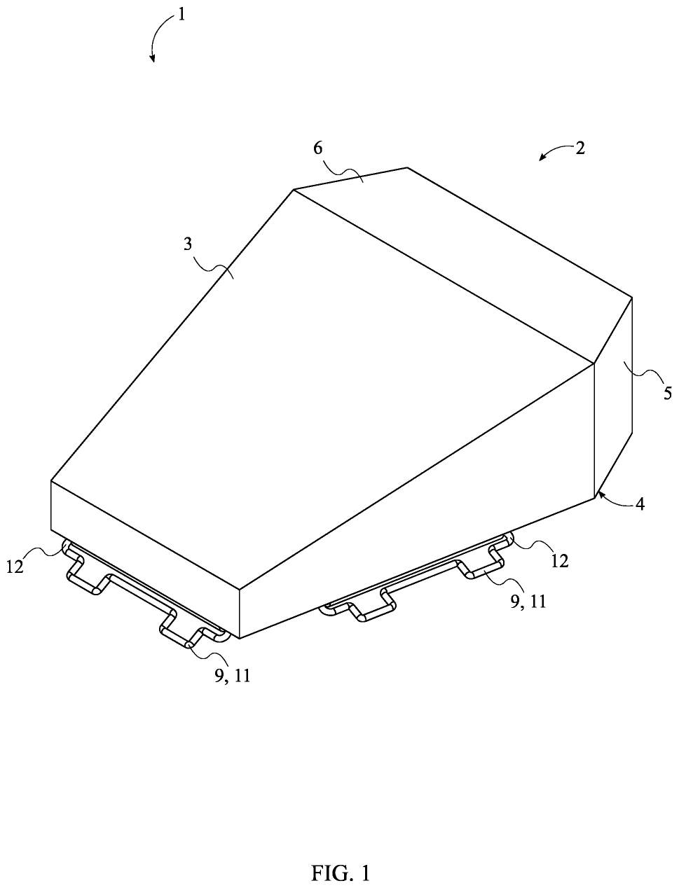

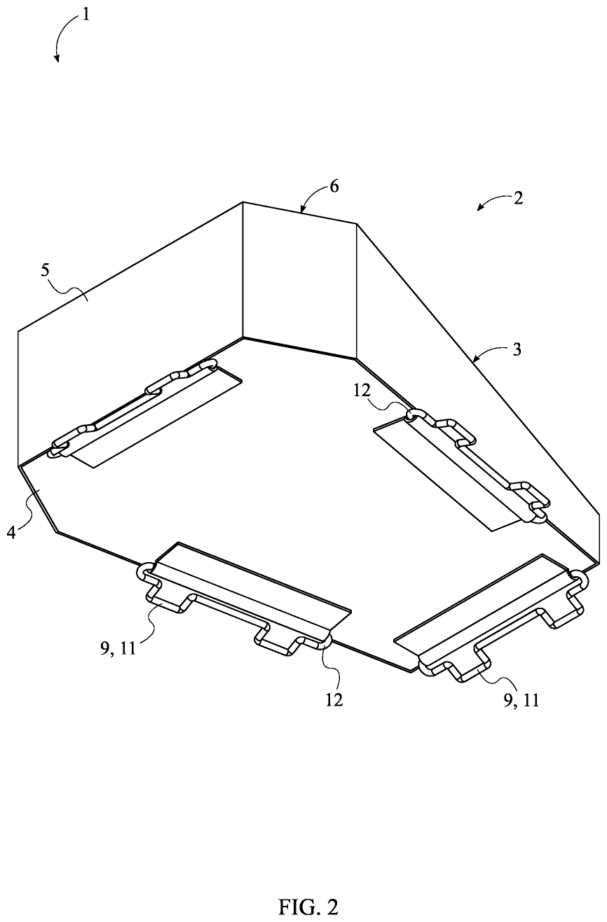

The present invention is a body limb support system that can be used to prop up different body parts during a tattoo session for greater comfort to the user and the tattoo artist and to facilitate the procedure. A preferred embodiment of the present invention comprises at least one limb pad 1. As can be seen in FIGS. 1 and 2, the at least one limb pad 1 is a versatile support device that can be arranged in multiple ways to support different body parts. For example, the at least one limb pad 1 can be used as a footrest or an armrest to support the leg or the arm of the user while the tattoo artist is working on the respective area. However, the at least one limb pad 1 can be utilized in different situations that require a body limb to be propped up for long periods of time.

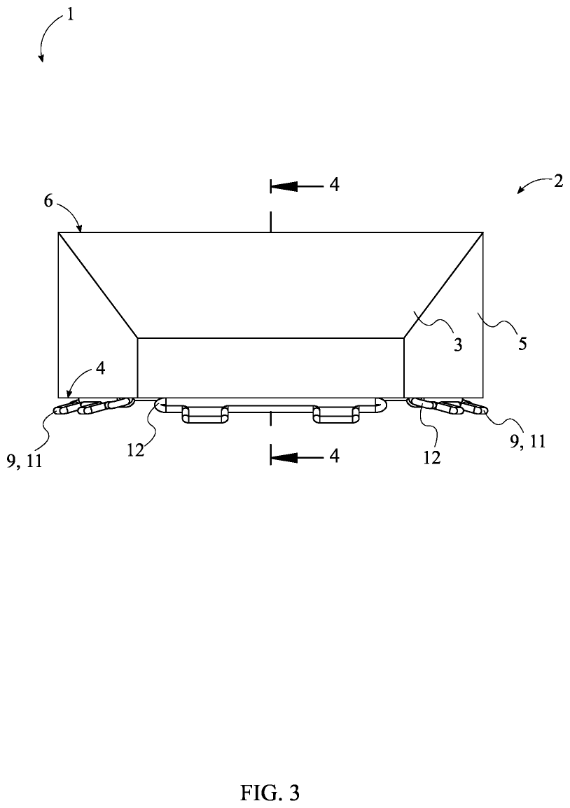

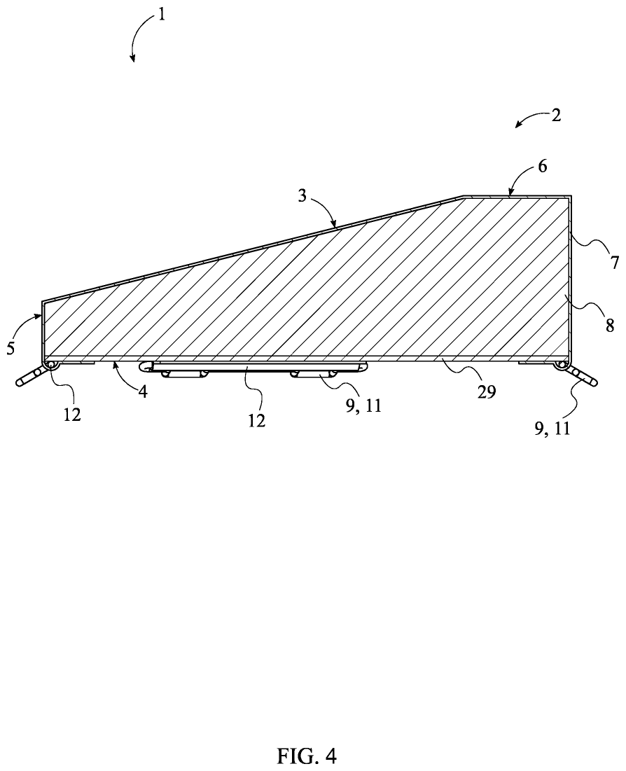

The general configuration of the aforementioned components enables a professional to comfortably and efficiently treat or perform on the target body area without the user or the professional being uncomfortable during the procedure. As can be seen in FIG. 1 through 4, the at least one limb pad 1 is designed to be comfortably positioned under the desired body area. The at least one limb pad 1 comprises a pad body 2 and a plurality of pad fasteners 9. The pad body 2 is preferably a soft structure that supports the target body area without causing damage to the body limb or the surface onto which the pad body 2 is placed. The plurality of pad fasteners 9 enables multiple units of the at least one limb pad 1 to be attached to each other in different configurations to support the desired body limb. The pad body 2 comprises a resting flat surface 3, a base flat surface 4, and a lateral surface 5 corresponding to the different surfaces surrounding the pad body 2. The resting flat surface 3 preferably corresponds to the surface where the body limb is placed on. The base flat surface 4 corresponds to the surface that is in contact with the object or surface onto which the pad body 2 is placed, such as a chair or table. Thus, the resting flat surface 3 and the base flat surface 4 are positioned opposite to each other about the pad body 2 to maintain the body limb elevated above the surface or object under the pad body 2. In addition, the resting flat surface 3 and the base flat surface 4 are oriented at an acute angle with each other so that the body limb comfortably rests on the pad body 2. The lateral surface 5 corresponds to the rest of the surfaces surrounding the pad body 2 that are formed from the thickness of the pad body 2. Thus, the lateral surface 5 is positioned in between the resting flat surface 3 and the base flat surface 4. Furthermore, the plurality of pad fasteners 9 is distributed around the lateral surface 5 so that multiple units of the at least one limb pad 1 can be attached to each other in different ways to accommodate different body areas. The plurality of pad fasteners 9 is perimetrically mounted to the base flat surface 4 so that each of the plurality of pad fasteners 9 does not interfere with the body limb resting on the pad body 2. In other embodiments, the plurality of pad fasteners 9 can be provided as detachable pad fasteners that can be rearranged in multiple ways.

The pad body 2 is designed to comfortably support different body parts so that the professional may work on the target body area without causing discomfort to the user or the professional. As can be seen in FIG. 1 through 4, the pad body 2 may further comprise a leveling flat surface 6 to increase the comfort to the user during long periods of time. The leveling flat surface 6 provides a resting place onto which the body limb can be placed on before reaching the resting flat surface 3. So, the leveling flat surface 6 is positioned adjacent to the resting flat surface 3. In addition, the leveling flat surface 6 and the base flat surface 4 are positioned opposite to each other about the pad body 2 to create a continuous surface between the resting flat surface 3 and the leveling flat surface 6. The leveling flat surface 6 is also positioned parallel to the base flat surface 4. Thus, the leveling flat surface 6 supports a portion of a body limb so that the portion of the body limb does not sharply fall off the peak of the resting flat surface 3, which reduces discomfort felt in the body limb. In other embodiments, the pad body 2 and the resting flat surface 3 may be shaped differently to accommodate specific body parts.

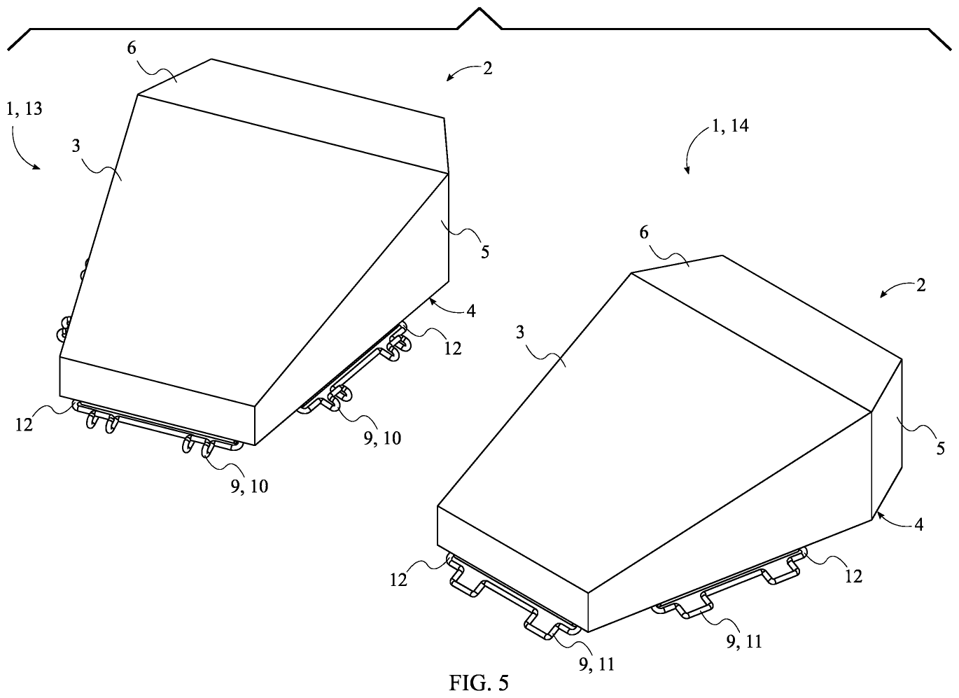

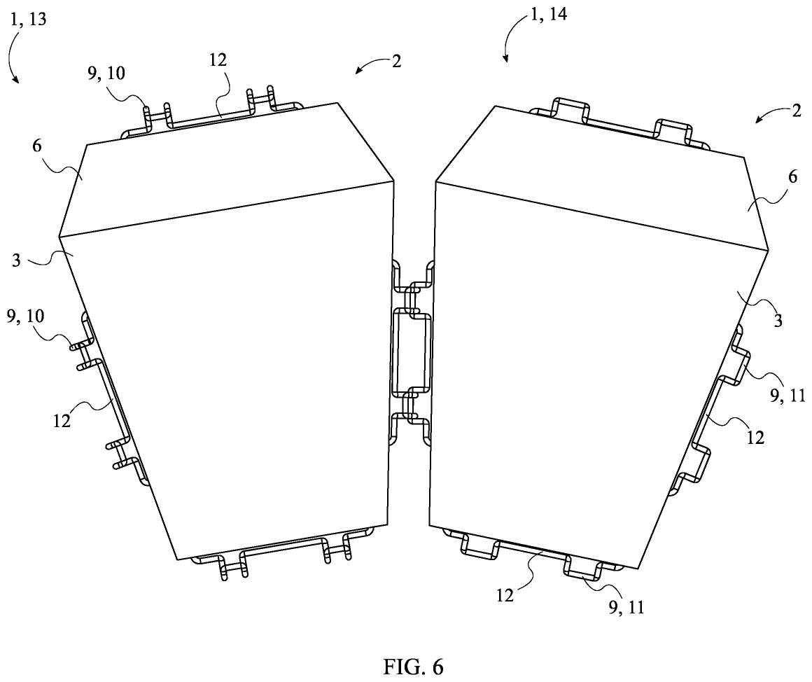

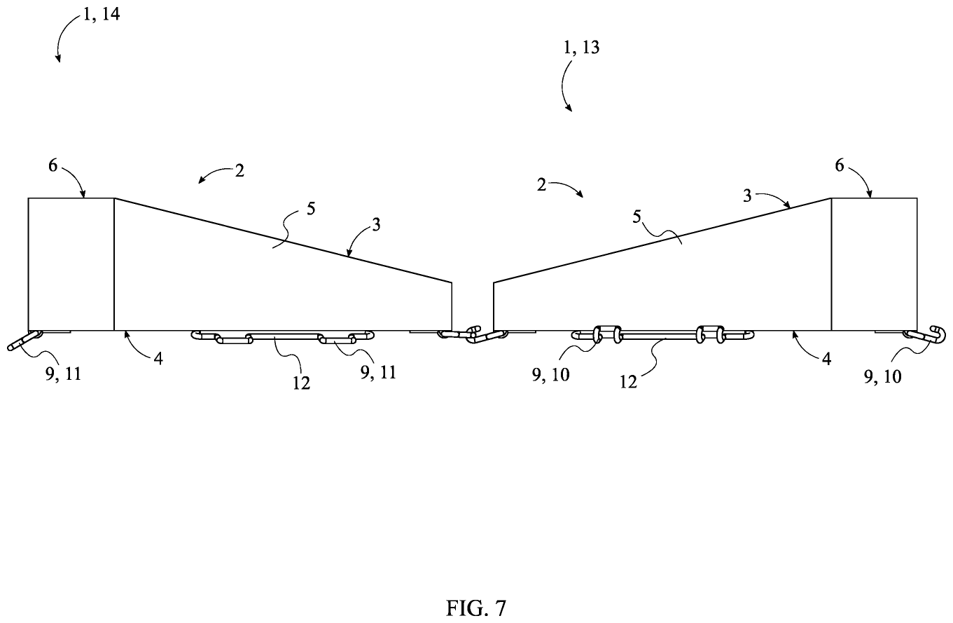

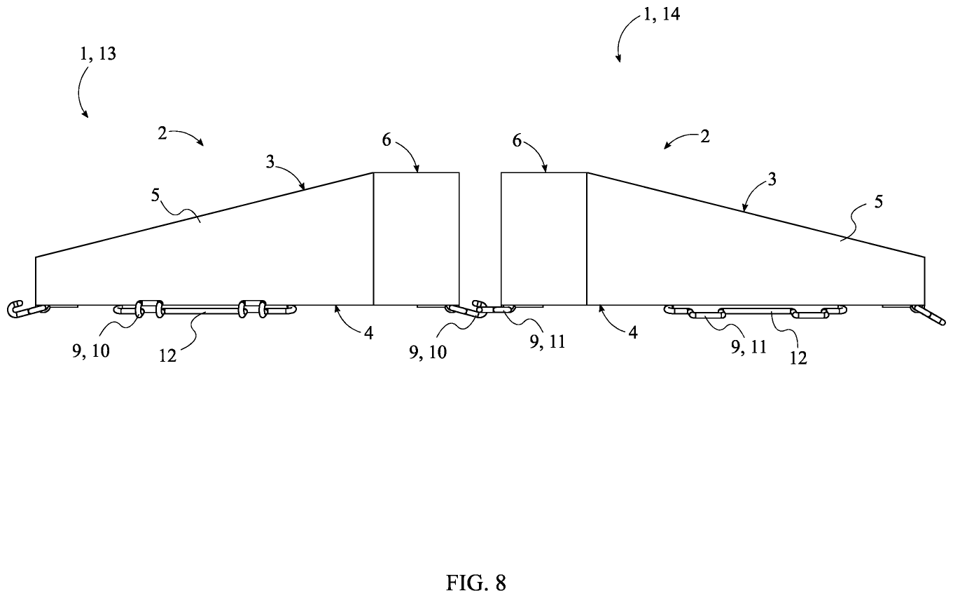

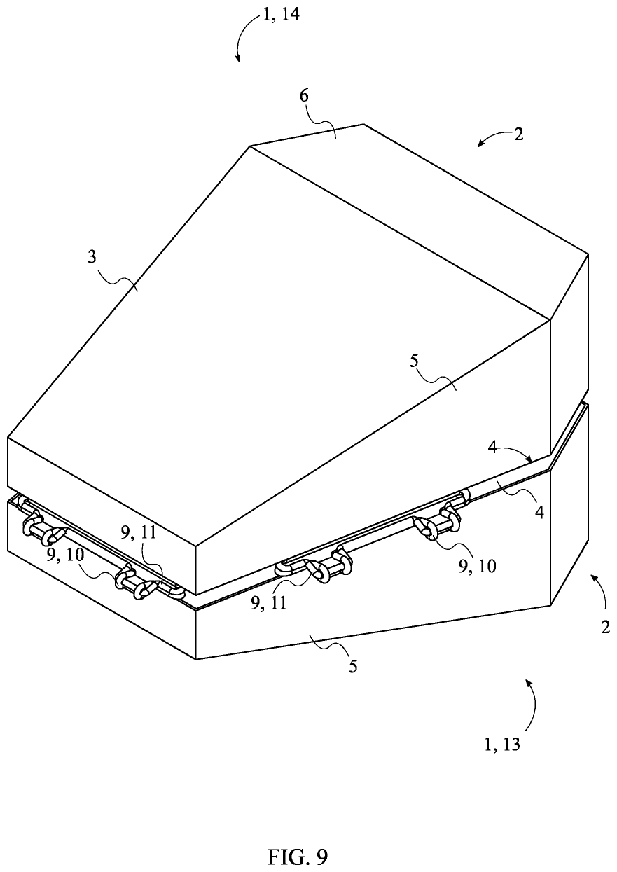

As previously discussed, multiple units of the at least one limb pad 1 can be attached to each other to form different support structures using the plurality of pad fasteners 9. As can be seen in FIG. 5 through 9, the at least one limb pad 1 may be a first limb pad 13 and a second limb pad 14 that can be put together to form a module support structure that can be arranged in multiple ways. The pad body 2 of the first limb pad 13 is positioned adjacent to the pad body 2 of the second limb pad 14 to form a single modular support structure. In addition, to secure the first limb pad 13 to the second limb pad 14, a selected pair of pad fasteners from the plurality of pad fasteners 9 of the first limb pad 13 is engaged to a selected pair of pad fasteners from the plurality of pad fasteners 9 of the second limb pad 14. Thus, the user can arrange the first limb pad 13 and the second limb pad 14 in different configurations to accommodate different body parts. For example, the first limb pad 13 can be connected to the second limb pad 14 sideways so that the first limb pad 13 and the second limb pad 14 are positioned side by side. In another configuration, the first limb pad 13 can be connected to the second limb pad 14 terminally so that the first limb pad 13 and the second limb pad 14 are connected in series. This connection in series can be done with the resting flat surface 3 of the first limb pad 13 positioned facing the resting flat surface 3 of the second limb pad 14, or the resting flat surface 3 of the first limb pad 13 and the resting flat surface 3 of the second limb pad 14 positioned facing away from each other. Further, in another configuration, the first limb pad 13 and the second limb pad 14 can be attached to each other to form a single structure by positioning the base flat surface 4 of the first limb pad 13 against the base flat surface 4 of the second limb pad 14 and engaging all the plurality of pad fasteners 9 of the first limb pad 13 with the corresponding pad fasteners of the plurality of pad fasteners 9 of the second limb pad 14. Thus, a single support structure is formed that elevates the desired body part even higher. In other embodiments, the first limb pad 13 and the second limb pad 14 may be arranged in different manners to accommodate different body parts.

Furthermore, to facilitate the different arrangements of the first limb pad 13 and the second limb pad 14, the plurality of pad fasteners 9 can be provided as pad fasteners that include matching fastening members that can be engaged with each other, as can be seen in FIG. 5. For example, each of the plurality of pad fasteners 9 of the first limb pad 13 is a hook 10, and each of the plurality of pad fasteners 9 of the second limb pad 14 is an eyelet 11. This allows for the first limb pad 13 to be releasably fastened to the second limb pad 14 while also enabling the first limb pad 13 to be hingedly connected to the second limb pad 14 using the multiple hooks and the multiple eyelets. In addition, each pad fastener of the plurality of pad fasteners 9 is preferably attached to the pad body 2 using a sheet of no-stretch vinyl fabric which enables each pad fastener to pivot individually or in pairs. In other embodiments, the plurality of pad fasteners 9 of the first limb pad 13 and the plurality of pad fasteners 9 of the second limb pad 14 can each be different matching type of fasteners. For example, the pad fasteners can be some combination of hook and loop fasteners, male and female fastener members, snap fasteners, etc.

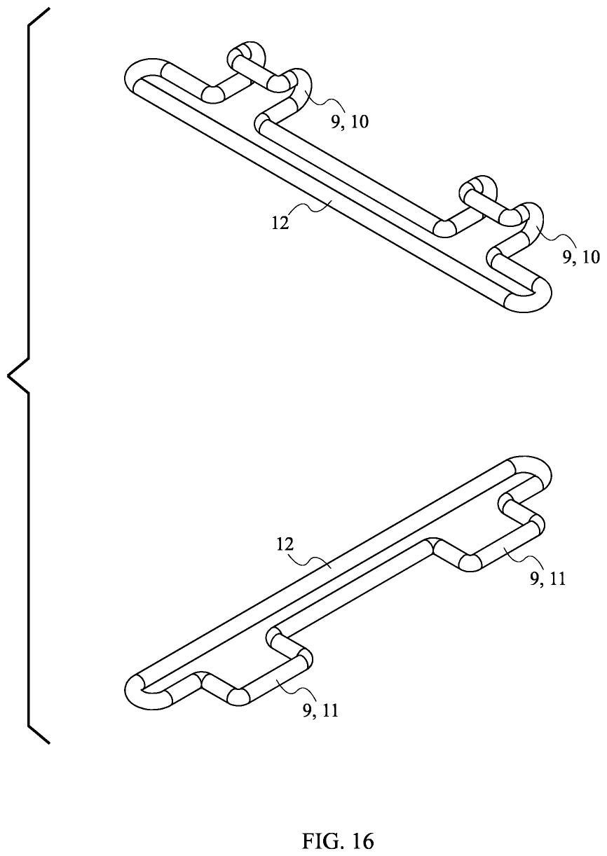

In some embodiments, the plurality of pad fasteners 9 can be provided in sections to facilitate the mounting of the plurality of pad fasteners 9 to the base flat surface 4. Moreover, the at least one limb pad 1 may further comprise a plurality of fastener members 12, as can be seen in FIGS. 1 through 4 and 16. Each fastener pair from the plurality of pad fasteners 9 is positioned offset from each other along a corresponding member from the plurality of fastener members 12 to ensure every side of the pad body 2 has at least one fastener pair. In addition, each fastener pair from the plurality of pad fasteners 9 is laterally connected to the corresponding member from the plurality of fastener members 12 to secure the fastener pairs to the corresponding fastener member. Further, each of the plurality of fastener members 12 is peripherally mounted onto the base flat surface 4 so that the corresponding fastener pair is laterally accessible to the user. In other embodiments, the plurality of pad fasteners 9 can be provided in different configurations to facilitate the attachment of other accessories to the pad body 2.

The pad body 2 is preferably designed to be used multiple times for different users, which requires the pad body 2 to be reusable and easily maintained. So, the pad body 2 may comprise a pad covering 7 and a pad cushion 8. As can be seen in FIG. 1 through 4, the pad cushion 8 provides the comfort to the body limb being supported by the pad body 2 while the pad covering 7 protects the pad cushion 8 and facilitates the cleaning of the pad body 2. For example, the pad cushion 8 may be made of foam or any other material that can provide a cushioning effect. The pad covering 7 may be made of any suitable bacterial-resistant fabric or leather to provide a non-slip and comfortable surface. In some embodiments, various indicia, logos, or graphic images may be applied to the pad covering 7 for personalization purposes or for promotional or advertising purposes. The pad cushion 8 is enclosed by the pad covering 7 so that the pad cushion 8 is not exposed to the surroundings of the pad body 2. In some embodiments, the pad body 2 may further comprise a rigid plate 29 to strengthen the structure of the pad body 2. The rigid plate 29 serves to provide additional structural strength but can be removed for additional comfort to the user laying on the pad body 2. The pad cushion 8 is mounted across the rigid plate 29 so that the rigid plate 29 supports the load being applied onto the pad cushion 8. The rigid plate 29 is also enclosed by the pad covering 7 so that the rigid plate 29 is not left exposed and does not damage the surface below. Finally, the resting flat surface 3 is positioned coincident with the pad cushion 8, while the base flat surface 4 is positioned coincident with the rigid plate 29. Therefore, the bottom of the pad body 2 provides additional support to the at least one limb pad 1. In another embodiment, the pad body 2 may also include a disposable cover made of a single-use material for sanitation purposes. The pad cushion 8 and the pad covering 7 are both enclosed by the disposable cover that can be easily removed after the procedure. A new disposable cover can be applied onto the pad covering 7 before each procedure to ensure the at least one limb pad 1 is clean and sanitized before use.

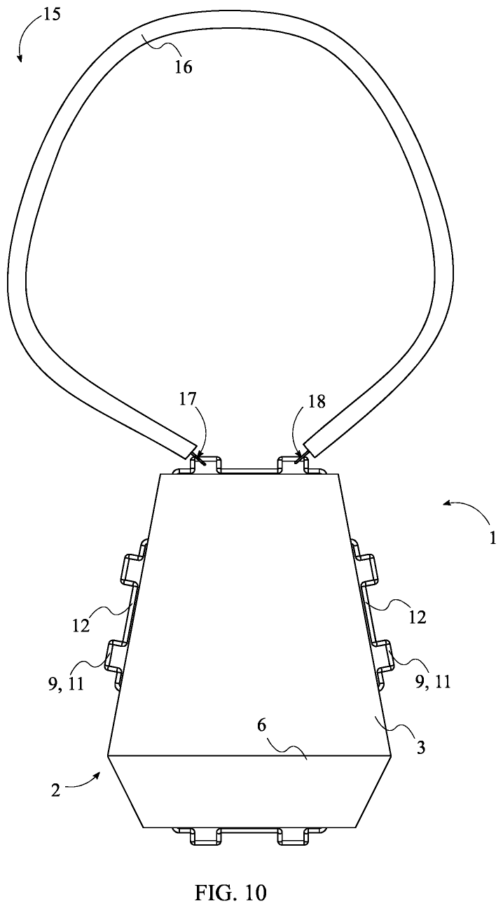

As previously mentioned, other accessories can be removably attached to the at least one limb pad 1 to facilitate the procedure. As can be seen in FIG. 10, the present invention may further comprise at least one support strap 15 that can be used to hang the at least one limb pad 1 from the user's body or from another external object. The at least one support strap 15 comprises a strap body 16, a first strap fastener 17, and a second strap fastener 18. The strap body 16 is preferably an elongated padded structure long enough to be hanged from the user's body or another object. The first strap fastener 17 and the second strap fastener 18 facilitate the removable attachment of the strap body 16 to the pad body 2. The first strap fastener 17 is terminally connected to the strap body 16. Similarly, the second strap fastener 18 is terminally connected to the strap body 16, opposite to the first strap fastener 17. This enables the at least one support strap 15 to be connected to the at least one limb pad 1 at the ends of the strap body 16, freeing the rest of the strap body 16 to be wrapped around the user's body or another object. For example, the strap body 16 can be wrapped around the user's shoulder so that the pad body 2 hangs under the user's arm. In addition, the strap body 16 is preferably fastened to the plurality of pad fasteners 9 adjacent to the resting flat surface 3 so that the leveling flat surface 6 rests under the user's armpit. This enables the rest of the user's arm to rest on the pad body 2 when the user must stay upright for long periods of time. To secure the strap body 16 to the pad body 2, the first strap fastener 17 is engaged to a first selected pad fastener from the plurality of pad fasteners 9. Likewise, the second strap fastener 18 is engaged to a second selected pad fastener from the plurality of pad fasteners 9. Thus, the pad body 2 securely hangs from the strap body 16. In other embodiments, the present invention may include other accessories that enable the at least one limb pad 1 to be secured to different body parts or different objects.

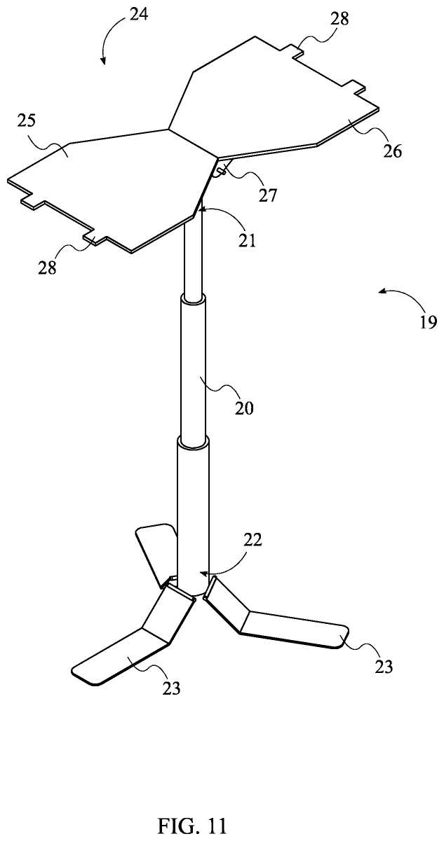

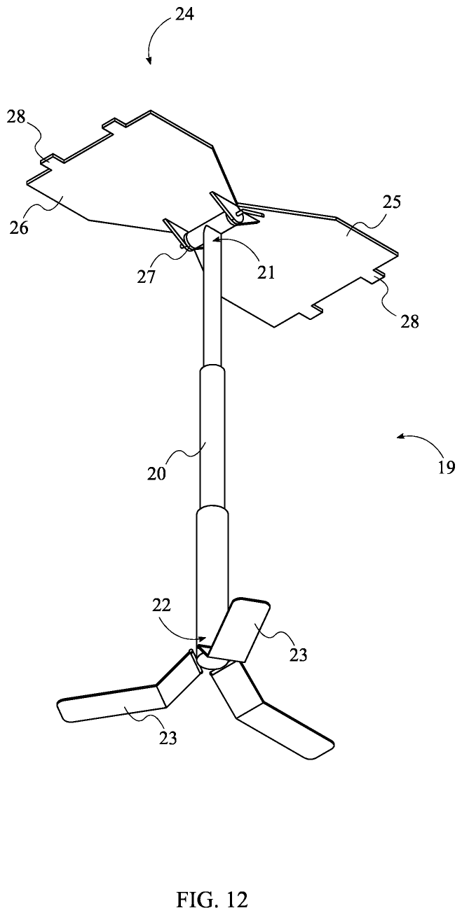

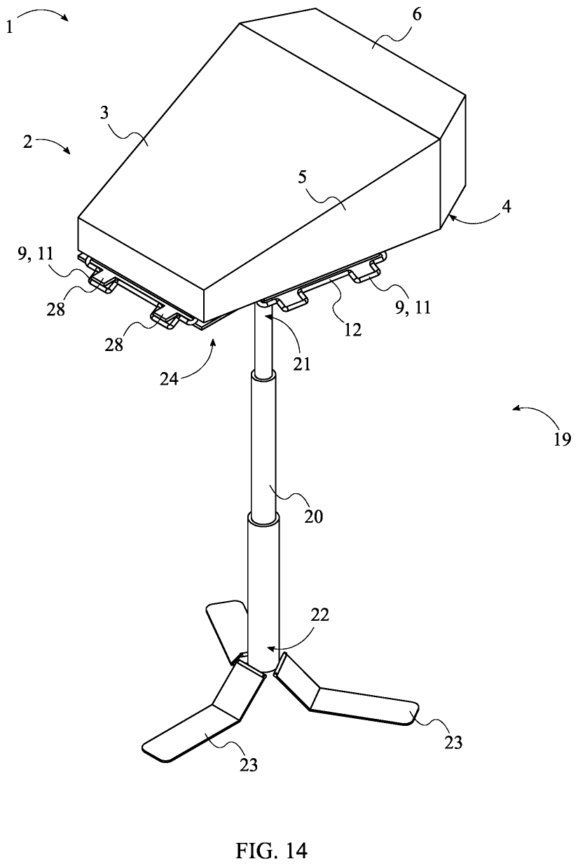

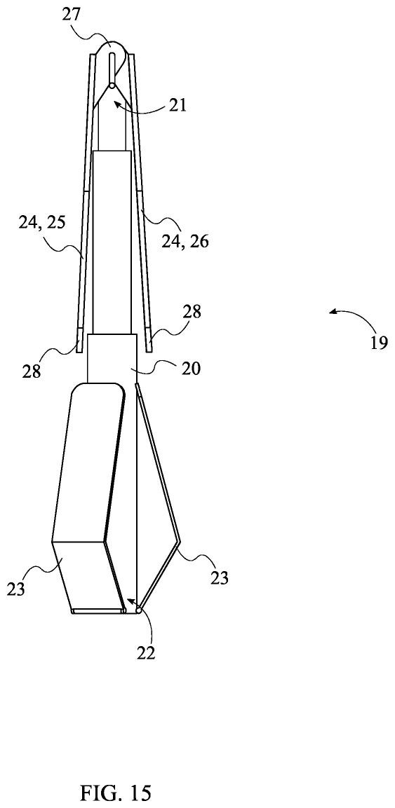

In case the at least one limb pad 1 cannot be supported on an object or elevated surface, the present invention may further comprise a support stand 19. As can be seen in FIG. 11 through 15, the support stand 19 enables the user to rest a body limb separate from the rest of the body. For example, the user can rest an arm separate from the body while laying on a seat or keep a leg up while seating down. The support stand 19 comprises a telescopic member 20, a plurality of foldable legs 23, a foldable platform 24, and a lockable hinge mechanism 27. The telescopic member 20 enables the support stand 19 to be enlarged or shortened to accommodate different heights. This enables the user to elevate the desired body limb to the desired height. The telescopic member 20 is preferably a tubular structure constructed from multiple sections of increasing diameter that telescopically engage with each other. For example, the telescopic member 20 can include three tubular sections of increasing diameter that are positioned concentric to each other. The three tubular sections are also telescopically engaged with each in increasing diameter size. The plurality of foldable legs 23 is designed to enable the user to deploy the support stand 19 when needed as well as to easily store away the support stand 19. Similarly, the foldable platform 24 serves to receive the at least one limb pad 1 while also enabling the easy deployment and storage of the support stand 19. The lockable hinge mechanism 27 enables the quick deployment and storage of the foldable platform 24 while ensuring that the foldable platform 24 stays in the desired position. Moreover, the telescopic member 20 may also comprise a locking mechanism that locks a working length of the telescopic member 20 in place. This prevents the telescopic member 20 from collapsing due to the load applied onto the foldable platform 24. In addition, the telescopic member 20 comprises a first member end 21 and a second member end 22 corresponding to the terminal ends of the tubular structure of the telescopic member 20. The foldable platform 24 is operatively mounted to the first member end 21 by the lockable hinge mechanism 27, wherein the lockable hinge mechanism 27 is used to selectively lock the foldable platform 24 in place. For example, the foldable platform 24 may include a first platform section 25 and a second platform section 26, and the lockable hinge mechanism 27 may be a pivot joint with a lock. The lock of the pivot joint can be a pull pin that engages with the pivot joint and can be easily pulled out and inserted into the pivot joint. Further, the pivot joint with a lock can be terminally mounted to the first member end 21. The first platform section 25 and the second platform section 26 are positioned opposite to each other across the pivot joint. Also, the first platform section 25 and the second platform section 26 can each be hingedly connected to the pivot joint. Thus, the user can easily deploy the foldable platform 24 by folding up the first platform section 25 and the second platform section 26 and then the user can lock the pivot joint in place. To store away the support stand 19, the user releases the pivot joint and folds down the first platform section 25 and the second platform section 26. In other embodiments, the foldable platform 24 and the lockable hinge mechanism 27 can be modified to accommodate different foldable mechanisms.

Furthermore, each of the plurality of foldable legs 23 is hingedly connected around the second member end 22 to provide a stable base for the support stand 19. As can be seen in FIG. 11 through 15, the plurality of foldable legs 23 can include three foldable legs distributed around the second member end 22 to form a tripod structure to support the load placed on the foldable platform 24. In addition, each of the plurality of foldable legs 23 is preferably a flat leg that sits flat on the ground or surface on which the support stand 19 is placed. The flat design of the plurality of foldable legs 23 increases the balance of the support stand 19 to prevent any accidental movement of the pad body 2 that may affect the procedure. A tripod hinge with a lock can be used to hingedly connect each of the plurality of foldable legs 23 to the telescopic member 20. Thus, the user can lock in place the plurality of foldable legs 23 without worrying that load applied onto the support stand 19 may cause the plurality of foldable legs 23 to collapse during use. The tripod hinge also enables the user to easily fold away the plurality of foldable legs 23 to quickly store away the support stand 19. Moreover, the telescopic member 20 is positioned normal to the foldable platform 24 to maintain the at least one limb pad 1 parallel to the ground. Then, to secure the at least one limb pad 1 to the support stand 19, the base flat surface 4 is positioned across the foldable platform 24, opposite to the first member end 21, so that the pad body 2 rests on the foldable platform 24. Finally, the pad body 2 is attached onto the foldable platform 24 by the plurality of pad fasteners 9 to secure the pad body 2 to the foldable platform 24.

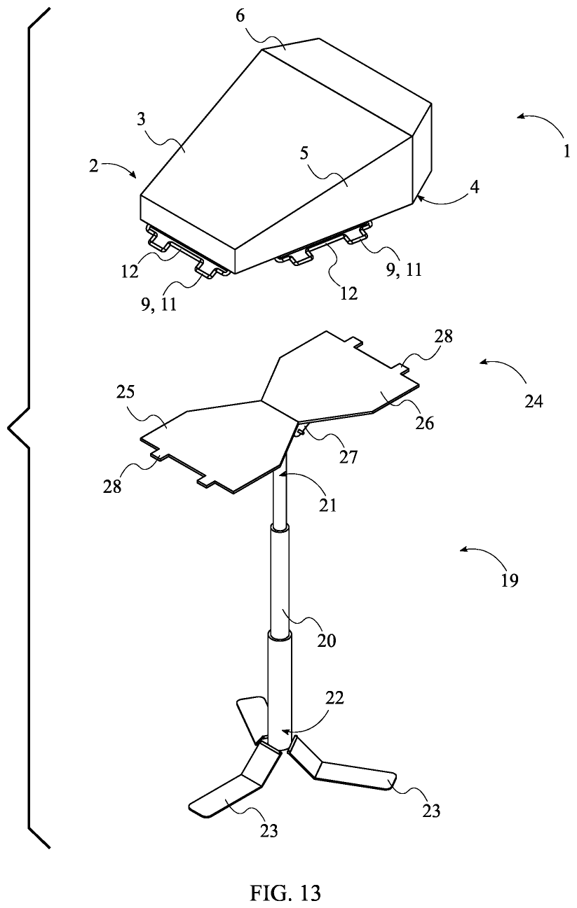

In some embodiments, the foldable platform 24 may provide means to engage the plurality of pad fasteners 9. As can be seen in FIG. 13 through 15, each of the plurality of pad fasteners 9 can be an eyelet 11, and the support stand 19 may further comprise a plurality of attachment tabs 28. Each of the plurality of attachment tabs 28 is designed to engage with the multiple eyelets to secure the pad body 2 to the foldable platform 24. To do so, the plurality of attachment tabs 28 is perimetrically connected around the foldable platform 24 so that multiple attachment tabs can be engaged with the eyelets on the pad body 2. In addition, the pad body 2 is locked in place by distributing the plurality of attachment tabs 28 around the foldable platform 24. Finally, to secure the pad body 2 to the foldable platform 24, each of the plurality of pad fasteners 9 is engaged to a corresponding tab from the plurality of attachment tabs 28, which prevent the pad body 2 from moving around the foldable platform 24. In other embodiments, different fastening mechanisms can be utilized to secure the pad body 2 to the foldable platform 24. Additionally, the support stand 19 may include other accessories such as an adjustable triple-hinged arm that can be clamped to the telescopic member 20 and reach out to the side, and up above the foldable platform 24, to hold a light source to illuminate the tattoo area. Moreover, the support stand 19 may include a wireless charging clip/holder for reference material, streaming live tattooing, and recording time-lapse videos. Alternatively, the support stand 19 may include a manual clip for paper reference material as well as an integrated power source to supply power to the light source and the wireless charger in the clip. Finally, the foldable platform 24 may be removed such that the support stand 19 can be used as to hold a light or as a phone holder.

Although the invention has been explained in relation to its preferred embodiment, it is to be understood that many other possible modifications and variations can be made without departing from the spirit and scope of the invention.

What is claimed is:

1. A body limb support system comprising:
at least one limb pad;
the at least one limb pad comprising a pad body and a plurality of pad fasteners;
the pad body comprising a resting flat surface, a base flat surface, and a lateral surface;
the resting flat surface and the base flat surface being positioned opposite to each other about the pad body;
the resting flat surface and the base flat surface being oriented at an acute angle with each other;
the lateral surface being positioned in between the resting flat surface and the base flat surface;
the plurality of pad fasteners being distributed around the lateral surface; and,
the plurality of pad fasteners being perimetrically mounted to the base flat surface.

2. The body limb support system as claimed in claim 1 comprising:
the pad body further comprising a leveling flat surface;
the leveling flat surface being positioned adjacent to the resting flat surface;
the leveling flat surface and the base flat surface being positioned opposite to each other about the pad body; and,
the leveling flat surface being positioned parallel to the base flat surface.

3. The body limb support system as claimed in claim 1 comprising:
the at least one limb pad being a first limb pad and a second limb pad;
the pad body of the first limb pad being positioned adjacent to the pad body of the second limb pad; and,
a selected pair of pad fasteners from the plurality of pad fasteners of the first limb pad being engaged to a selected pair of pad fasteners from the plurality of pad fasteners of the second limb pad.

4. The body limb support system as claimed in claim 3, wherein each of the plurality of pad fasteners of the first limb pad is a hook, and wherein each of the plurality of pad fasteners of the second limb pad is an eyelet.

5. The body limb support system as claimed in claim 1 comprising:
the at least one limb pad further comprising a plurality of fastener members;
each fastener pair from the plurality of pad fasteners being positioned offset from each other along a corresponding member from the plurality of fastener members;
each fastener pair from the plurality of pad fasteners being laterally connected to the corresponding member from the plurality of fastener members; and,
each of the plurality of fastener members being peripherally mounted onto the base flat surface.

6. The body limb support system as claimed in claim 1 comprising:
the pad body further comprising a pad covering and a pad cushion; and,
the pad cushion being enclosed by the pad covering.

7. The body limb support system as claimed in claim 1 comprising:
at least one support strap;
the at least one support strap comprising a strap body, a first strap fastener, and a second strap fastener;
the first strap fastener being terminally connected to the strap body;
the second strap fastener being terminally connected to the strap body, opposite to the first strap fastener;
the first strap fastener being engaged to a first selected pad fastener from the plurality of pad fasteners; and
the second strap fastener being engaged to a second selected pad fastener from the plurality of pad fasteners.

8. The body limb support system as claimed in claim 1 comprising:
a support stand;
the support stand comprising a telescopic member, a plurality of foldable legs, a foldable platform, and a lockable hinge mechanism;
the telescopic member comprises a first member end and a second member end;
the foldable platform being operatively mounted to the first member end by the lockable hinge mechanism, wherein the lockable hinge mechanism is used to selectively lock the foldable platform in place;
each of the plurality of foldable legs being hingedly connected around the second member end;
the telescopic member being positioned normal to the foldable platform;
the base flat surface being positioned across the foldable platform, opposite to the first member end; and,
the pad body being attached onto the foldable platform by the plurality of pad fasteners.

9. The body limb support system as claimed in claim 8 comprising:
each of the plurality of pad fasteners being an eyelet;
the support stand further comprising a plurality of attachment tabs;
the plurality of attachment tabs being perimetrically connected around the foldable platform; and,
each of the plurality of pad fasteners being engaged to a corresponding tab from the plurality of attachment tabs.

10. A body limb support system comprising:
at least one limb pad;
the at least one limb pad comprising a pad body and a plurality of pad fasteners;
the pad body comprising a resting flat surface, a base flat surface, a lateral surface, a leveling flat surface, a pad covering, and a pad cushion;
the resting flat surface and the base flat surface being positioned opposite to each other about the pad body;
the resting flat surface and the base flat surface being oriented at an acute angle with each other;
the lateral surface being positioned in between the resting flat surface and the base flat surface;
the leveling flat surface being positioned adjacent to the resting flat surface;
the leveling flat surface and the base flat surface being positioned opposite to each other about the pad body;
the leveling flat surface being positioned parallel to the base flat surface;
the plurality of pad fasteners being distributed around the lateral surface;
the plurality of pad fasteners being perimetrically mounted to the base flat surface; and,
the pad cushion being enclosed by the pad covering.

11. The body limb support system as claimed in claim 10 comprising:
the at least one limb pad being a first limb pad and a second limb pad;
the pad body of the first limb pad being positioned adjacent to the pad body of the second limb pad; and,
a selected pair of pad fasteners from the plurality of pad fasteners of the first limb pad being engaged to a selected pair of pad fasteners from the plurality of pad fasteners of the second limb pad.

12. The body limb support system as claimed in claim 11, wherein each of the plurality of pad fasteners of the first limb pad is a hook, and wherein each of the plurality of pad fasteners of the second limb pad is an eyelet.

13. The body limb support system as claimed in claim 10 comprising:
the at least one limb pad further comprising a plurality of fastener members;
each fastener pair from the plurality of pad fasteners being positioned offset from each other along a corresponding member from the plurality of fastener members;
each fastener pair from the plurality of pad fasteners being laterally connected to the corresponding member from the plurality of fastener members; and,
each of the plurality of fastener members being peripherally mounted onto the base flat surface.

14. The body limb support system as claimed in claim 10 comprising:
at least one support strap;
the at least one support strap comprising a strap body, a first strap fastener, and a second strap fastener;
the first strap fastener being terminally connected to the strap body;
the second strap fastener being terminally connected to the strap body, opposite to the first strap fastener;
the first strap fastener being engaged to a first selected pad fastener from the plurality of pad fasteners; and,
the second strap fastener being engaged to a second selected pad fastener from the plurality of pad fasteners.

15. The body limb support system as claimed in claim 10 comprising:
a support stand;
each of the plurality of pad fasteners being an eyelet;
the support stand comprising a telescopic member, a plurality of foldable legs, a foldable platform, a lockable hinge mechanism, and a plurality of attachment tabs;
the telescopic member comprises a first member end and a second member end;
the foldable platform being operatively mounted to the first member end by the lockable hinge mechanism, wherein the lockable hinge mechanism is used to selectively lock the foldable platform in place;
each of the plurality of foldable legs being hingedly connected around the second member end;
the telescopic member being positioned normal to the foldable platform;
the base flat surface being positioned across the foldable platform, opposite to the first member end;
the pad body being attached onto the foldable platform by the plurality of pad fasteners;
the plurality of attachment tabs being perimetrically connected around the foldable platform; and,
each of the plurality of pad fasteners being engaged to a corresponding tab from the plurality of attachment tabs.

16. A body limb support system comprising:
at least one limb pad;
the at least one limb pad comprising a pad body, a plurality of pad fasteners, and a plurality of fastener members;
the pad body comprising a resting flat surface, a base flat surface, a lateral surface, a leveling flat surface, a pad covering, and a pad cushion;
the resting flat surface and the base flat surface being positioned opposite to each other about the pad body;
the resting flat surface and the base flat surface being oriented at an acute angle with each other;
the lateral surface being positioned in between the resting flat surface and the base flat surface;
the leveling flat surface being positioned adjacent to the resting flat surface;
the leveling flat surface and the base flat surface being positioned opposite to each other about the pad body;
the leveling flat surface being positioned parallel to the base flat surface;
the plurality of pad fasteners being distributed around the lateral surface;
the plurality of pad fasteners being perimetrically mounted to the base flat surface;
the pad cushion being enclosed by the pad covering;
each fastener pair from the plurality of pad fasteners being positioned offset from each other along a corresponding member from the plurality of fastener members;
each fastener pair from the plurality of pad fasteners being laterally connected to the corresponding member from the plurality of fastener members; and,
each of the plurality of fastener members being peripherally mounted onto the base flat surface.

17. The body limb support system as claimed in claim 16 comprising:
the at least one limb pad being a first limb pad and a second limb pad;
the pad body of the first limb pad being positioned adjacent to the pad body of the second limb pad; and,
a selected pair of pad fasteners from the plurality of pad fasteners of the first limb pad being engaged to a selected pair of pad fasteners from the plurality of pad fasteners of the second limb pad.

18. The body limb support system as claimed in claim 17, wherein each of the plurality of pad fasteners of the first limb pad is a hook, and wherein each of the plurality of pad fasteners of the second limb pad is an eyelet.

19. The body limb support system as claimed in claim 16 comprising:
at least one support strap;
the at least one support strap comprising a strap body, a first strap fastener, and a second strap fastener;
the first strap fastener being terminally connected to the strap body;
the second strap fastener being terminally connected to the strap body, opposite to the first strap fastener;
the first strap fastener being engaged to a first selected pad fastener from the plurality of pad fasteners; and,
the second strap fastener being engaged to a second selected pad fastener from the plurality of pad fasteners.

20. The body limb support system as claimed in claim 16 comprising:
a support stand;
each of the plurality of pad fasteners being an eyelet;
the support stand comprising a telescopic member, a plurality of foldable legs, a foldable platform, a lockable hinge mechanism, and a plurality of attachment tabs;
the telescopic member comprises a first member end and a second member end;
the foldable platform being operatively mounted to the first member end by the lockable hinge mechanism, wherein the lockable hinge mechanism is used to selectively lock the foldable platform in place;
each of the plurality of foldable legs being hingedly connected around the second member end;
the telescopic member being positioned normal to the foldable platform;
the base flat surface being positioned across the foldable platform, opposite to the first member end;
the pad body being attached onto the foldable platform by the plurality of pad fasteners;
the plurality of attachment tabs being perimetrically connected around the foldable platform; and,
each of the plurality of pad fasteners being engaged to a corresponding tab from the plurality of attachment tabs.
